(19) United States
(12) Patent Application Publication
(10) Pub. No.: US 2004/0172024 A1
(43) Pub. Date: Sep. 2, 2004

(54) System and method for stabilizing the spine by securing spine stabilization rods in crossed disposition

(76) Inventor: Josef E. Gorek, Larkspur, CA (US)

Correspondence Address: Joseph P. Errico, 150 Douglas Road, Far Hills, NJ, 07931, US

(21) Appl. No.: 10/793,118

(22) Filed: Mar. 4, 2004

Related U.S. Application Data
(63) Continuation of application No. 10/200,820, filed on Jul. 22, 2002, now abandoned.

(51) Int. Cl. A61B 17/56

(52) U.S. Cl. 606/61

(57) ABSTRACT

A spine stabilization assembly includes two rods and a cross link for compression locking the rods in crossed disposition to stabilize a spine. The rods optionally have flattened central portions with ridges to enhance the compression lock. The cross link in one embodiment is a bolt with a trough that receives both rods for relative angulation and the bolt receives a nut that when threaded on the bore compresses one rod against the other in the trough. In another embodiment, the cross link is a split sphere with halves that can rotate with respect to one another, and each half can receive a rod for angulation. A clamp secured around the sphere compresses the assembly to lock the rods in crossed disposition. Another embodiment features rods with enlarged central portions having a bore, and a screw passing through one bore and threading in another compresses the rods together.

### CROSS REFERENCE TO RELATED APPLICATION

This application is a continuing application of U.S. patent application Ser. No. 10/200,820 filed Jul. 22, 2002 and entitled “System and Method for Stabilizing the Spine by Securing Spine Stabilization Rods in Crossed Disposition”.

### FIELD OF THE INVENTION

This invention relates generally to system and methods for stabilizing the spine and more specifically to a system and method for securing spine stabilization rods in crossed disposition.

### BACKGROUND OF THE INVENTION

The bones and connective tissue of an adult human spinal column consists of more than twenty discrete bones coupled sequentially to one another by a tri-joint complex which consists of an anterior disc and the two posterior facet joints, the anterior discs of adjacent bones being cushioned by cartilage spacers referred to as intervertebral discs. These more than twenty bones are anatomically categorized as being members of one of four classifications: cervical, thoracic, lumbar, or sacral. The cervical portion of the spine, which comprises the top of the spine, up to the base of the skull, includes the first seven vertebrae. The intermediate twelve bones are the thoracic vertebrae, and connect to the lower spine comprising the five lumbar vertebrae. The base of the spine is the sacral bones (including the coccyx). The component bones of the cervical spine are generally smaller than those of the thoracic and lumbar spine.

The spinal column of bones is highly complex in that it includes over twenty bones coupled to one another, housing and protecting critical elements of the nervous system having innumerable peripheral nerves and circulatory bodies in close proximity. In spite of these complexities, the spine is a highly flexible structure, capable of a high degree of curvature and twist in nearly every direction. Genetic or developmental irregularities, trauma, chronic stress, tumors, and disease, however, can result in spinal pathologies which either limit this range of motion, or which threaten the critical elements of the nervous system housed within the spinal column. A variety of systems have been disclosed in the art that achieve this immobilization by implanting artificial assemblies in or on the spinal column. These assemblies may be classified as anterior, posterior, or lateral implants. As the classifications suggest, lateral and anterior assemblies are coupled to the anterior portion of the spine, which is the sequence of vertebral bodies. Posterior implants generally comprise pairs of rods, which are aligned along the axis which the bones are to be disposed, and which are then attached to the spinal column by either hooks which couple to the lamina or attach to the transverse processes, or by screws which are inserted through the pedicles.

As is known in the art, stabilization assemblies utilizing two spinal rods fastened in parallel on either side of the spinous process can be strengthened by using one or more rods to transversely bridge the parallel rods. Such transverse connectors typically consist of rods, plates, and bars linked to the parallel rods by coupling mechanisms with set screws, nuts, or a combination of each. Examples of existing systems include the Transverse Link Device and Crosslink manufactured by Sofamor Danek, the Trans-Connector manufactured by Synthes, and the Modular Cross Connector and Transverse Rod Connector manufactured by AcroMed.

While such transverse connectors are useful in certain applications for increasing the construct strength and more equally distributing stresses, there are many conditions which preclude the use of such connectors. For example, with degenerative spondylolisthesis, the L-4 and L-5 screw heads are too close together and consequently there is not enough space on the rod to apply a transverse connector. In addition, transverse connecting constructs presently in use suffer from several drawback. First, many are unstable in that they are routinely subject to translational shifting from right to left and rotation about the screw axis. Second, the laminectomy site remains uncovered. Third, the transverse rods typically are placed over the bone graft sites and the facet joints, which interferes with muscle apposition that is necessary for vascular supply to the bone graft, and makes radiographic assessment of the fusion mass more difficult even when oblique radiographs are used.

Accordingly, there is a need for an improved system and method for stabilizing immobilization assemblies for the spine.

Therefore, it is an object of the present invention to provide a system and method for stabilizing the spine by securing spine stabilization rods in crossed disposition.

It is another object of the present invention to provide a cross link for securing at least two rods in crossed disposition, wherein at least one of the rods can be angulated relative to the other rod, and wherein the rods can thereafter be secured in cross disposition.

It is yet another object of the present invention to provide a cross link for securing at least two rods in crossed disposition, wherein at least one of the rods can be angulated relative to the other rod, and wherein the rods can thereafter be secured in cross disposition, and wherein at least one of the rods remains free to move longitudinally after the rods are secured in crossed disposition.

It is still another object of the present invention to provide a cross link for securing at least two rods in crossed disposition, wherein at least one of the rods can be angulated relative to the other rod, and wherein the rods can thereafter be secured in cross disposition, and wherein the angle at which at least one of the rods is angulated with respect to the other rod is compressively fixable.

It is still another object of the present invention to provide a cross link for securing at least two rods in crossed disposition, wherein at least one of the rods can be angulated relative to the other rod, and wherein the rods can thereafter be secured in cross disposition, and wherein at least one of the rods in crossed disposition can be compression locked to the cross link.

It is a further object of the present invention to provide a cross link for securing at least two rods in crossed disposition, wherein the rods have centrally located features to facilitate their compression together.

It is a still further object of the present invention to provide a cross link for securing at least two rods in crossed disposition, wherein the rods have centrally located features to facilitate their compression together, and wherein the features include flattened surfaces and/or protrusions and/or laterally enlarged portions.

It is another still further object of the present invention to provide a cross link for securing at least two rods in crossed disposition, wherein the rods have centrally located features to facilitate their compression together, and wherein the features include a bore hole receiving a screw.

Other objects of the present invention not explicitly stated will be set forth and will be more clearly understood in conjunction with the descriptions of the preferred embodiments disclosed hereafter.

### SUMMARY OF THE INVENTION

The preceding objects of the invention are achieved by the present invention which provides a cross link for securing rods in crossed disposition.

More particularly, in a first embodiment the cross link includes a rod supporting body, for example, a bolt. The bolt has two extensions, for example, arms defining a trough that accepts a lower rod and an upper rod on top of the lower rod. The walls of the upper portion of the trough are formed so that a plurality of upper rod positions are possible, at least one of which is a position in which the upper rod is angulated relative to the lower rod. The surgeon can therefore sweep the upper rod through the plurality of angles until the desire angle is reached. The cross link further includes a compression providing element, for example, a nut that threads around the arms. Therefore, after the desired angle is reached, the nut can be tightened down onto the upper rod to compression lock the upper rod against the lower rod, and the lower rod against the floor of the trough. In this manner, the rods can be secured in crossed disposition.

In a second embodiment, each of the rods has as a securing feature a flattened central portion against which the other rod is to be compressed. The flattened surface is a securing feature in that it provides a greater amount of surface area against which the other rod can be compressed when locked in the cross link, compared to the merely tangential surface area provided by a fully cylindrical rod. The cross link bolt suitable for use with these alternate rods has a shallower lower portion of the trough than the bolt of the first embodiment, to accommodate the lower rod having the flattened portion, in that the flattened portion is more narrow than the other portions of the lower rod.

In a third embodiment, each of the rods has as a securing feature a flattened central portion, with radially extending ridges, against which the other rod is to be compressed. The ridges will interlock when the rods are compressed against one another at this central portion, providing even greater fixation than a fully cylindrical rod without ridges. Again, the cross link bolt suitable for use with these other alternate rods has a shallower lower portion of the trough than the bolt of the first embodiment, to accommodate the lower rod having the flattened portion and ridges, in that the flattened portion with ridges is more narrow than the other portions of the lower rod.

In a fourth embodiment, the rods again have as a securing feature a flattened central portion, with radially extending ridges, but the central portion is laterally extended beyond the boundaries of the cylinder defined by the rod body. This further increases the surface area against which the other rod can be compressed. Further, rather than a bolt and nut assembly to compress the rods together, this embodiment includes bores through the central portion of each rod. The bore of the lower rod is threaded, while the bore of the upper rod is smooth. A screw is provided for passing through the upper bore and thereafter threading in the lower bore to bring the rods together until. Accordingly, as the central regions come together, the ridges interlock, so that as the regions are compressed against one another, a compression lock is secured with a fixation greater than that achieved by compressing together traditional rods.

In a fifth embodiment, a rod supporting body, for example, a dual rod holder, in cooperation with a compression providing element, for example, a clamp, is adapted to hold rods in crossed disposition. The dual rod holder includes two body portions, for example, two hemispherical members rotationally mounted to one another at their flat sides, so that they collectively form a sphere that has hemispherical sections that can swivel relative to one another about a polar axis of the sphere. Each of the hemispheres has a rod receiving channel in which the rods can be placed. Once the rods are placed in the channels, they can be angled with respect to one another by a relative rotation of the hemispheres. Once the desired angle has been reached for the particular clinical application, the clamp applied around the hemispheres to encompass the rods fixes the hemispheres at their positions relative to one another, and fixes the rods in the channels so that they are secured in cross disposition.

In accordance with a method of the present invention, an immobilization assembly can be stabilized on the spine, preferably using the devices described. As one example of an appropriate clinical application in which the system and method of the present invention is useful, a patient presenting an L-4/L-5 degenerative spondylolisthesis receives the necessary laminectomy, but the stabilization rods, rather than being positioned in parallel on either side of the spinous process and being connected by a transverse connector, are set in crossed disposition. When necessary, this can be facilitated by the use of polyaxial screws with one pair of screws left relatively proud to allow the rod they support to cross over the other rod. A cross link of the present invention is applied to the rods before or after the setting of the rods in crossed disposition. Depending on the type of cross link used, it will be easier for the surgeon to apply the cross link to the rods before setting them, or to apply the cross link to the rods after setting them. In the case where the cross link is formed by modified rods (e.g., as in the fourth embodiment described above), the rods would typically be set in crossed disposition, then compressed together (e.g., by applying the screw to the bored central portions of the rods) to stabilize the construct. The soft tissues of the back can be sutured down onto the rods, which minimizes dead space and therefore reduces post-operation bleeding. Advantages of the method of the present invention include that the resulting fusion is easier to grade radiographically because the rods do not hide the area of interest, and that if reoperation is required, scar revision is easier because a depth and plane of resection are already defined. While the procedure has been described for use with the laminectomized spine, the system and method of the present invention are useful in other clinical applications as well. For example, many lumbar spinal fusions are performed for degenerative conditions, which often require a laminectomy. Thus, the present invention can be applied universally to all implant systems. As described below, single and multiple level constructs requiring in situ fixation are among the preferred applications.

While there has been described and illustrated specific embodiments of an intervertebral spacer device, it will be apparent to those skilled in the art that variations and modifications are possible without deviating from the broad spirit and principle of the present invention. The invention, therefore, shall not be limited to the specific embodiments discussed herein.

### BRIEF DESCRIPTION OF THE DRAWINGS

FIG. 1A is a schematic diagram of a plurality of vertebrae secured relative to one another by crossed rods being maintained in their crossed disposition by a cross link in accordance with the present invention.

FIG. 1B is a schematic diagram of a plurality of vertebrae secured relative to one another by an alternate arrangement of crossed rods being maintained in their cross disposition by a plurality of cross links in accordance with the present invention.

FIG. 2 is a perspective view of a first embodiment of the cross link shown in FIGS. 1A and 1B, maintaining two rods in crossed disposition.

FIG. 3 is a perspective view of a bolt of the cross link embodiment shown in FIG. 2.

FIG. 4 is a perspective view of a nut of the cross link embodiment shown in FIG. 2.

FIG. 5 is a perspective view of alternate lower and upper rods in accordance with a second embodiment of the cross link shown in FIGS. 1A and 1B, each having a securing feature on a surface against which the other rod is to be compressed.

FIG. 6 is a perspective view of the alternate lower and upper rods of FIG. 5, being secured in crossed disposition by an alternate bolt formed similarly to the bolt of FIG. 3, but having a different shelf height.

FIG. 7 is a perspective view of other alternate lower and upper rods in accordance with a third embodiment of the cross link shown in FIGS. 1A and 1B, each having a securing feature on a surface against which the other rod is to be compressed.

FIG. 8 is a perspective view of the other alternate lower and upper rods of FIG. 7, being secured in crossed disposition by another alternate bolt formed similarly to the bolt of FIG. 3, but having a different shelf height.

FIG. 9 is a perspective view of still other alternate lower and upper rods in accordance with a fourth embodiment of the cross link shown in FIGS. 1A and 1B, each having a securing feature on a surface against which the other rod is to be compressed.

FIG. 10 is a perspective view of the still other alternate lower and upper rods of FIG. 9, being secured in crossed disposition by a set screw passing through their enlarged central circular regions.

FIGS. 11A and 11B are perspective views of a dual rod holder and a cooperating clamp, respectively, in accordance with a fifth embodiment of the cross link shown in FIGS. 1A and 1B, which cooperate to hold rods in crossed disposition.

FIG. 12 is a perspective view of the fifth embodiment showing the dual rod holder of FIG. 11A and the clamp of FIG. 11B holding rods in crossed disposition.

FIG. 13 is a side cutaway view of an alternate dual rod holder of the fifth embodiment, showing an exemplary tension bearing rotational mounting.

### DETAILED DESCRIPTION OF THE PREFERRED EMBODIMENT

While the present invention will be described more fully hereinafter with reference to the accompanying drawings, in which particular embodiments and methods of implantation are shown, it is to be understood at the outset that persons skilled in the art may modify the invention herein described while achieving the functions and results of this invention. Accordingly, the descriptions that follow are to be understood as illustrative and exemplary of specific structures, aspects and features within the broad scope of the present invention and not as limiting of such broad scope. Like numbers refer to similar features of like elements throughout.

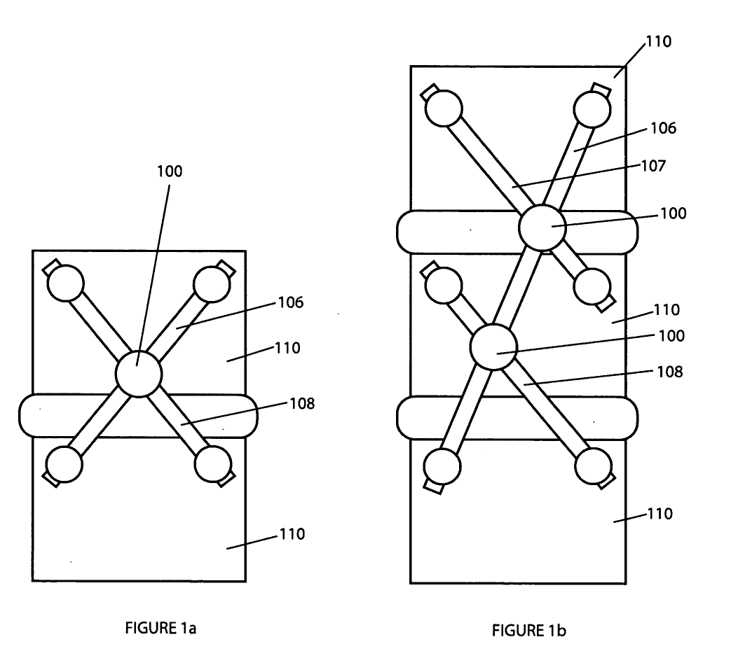

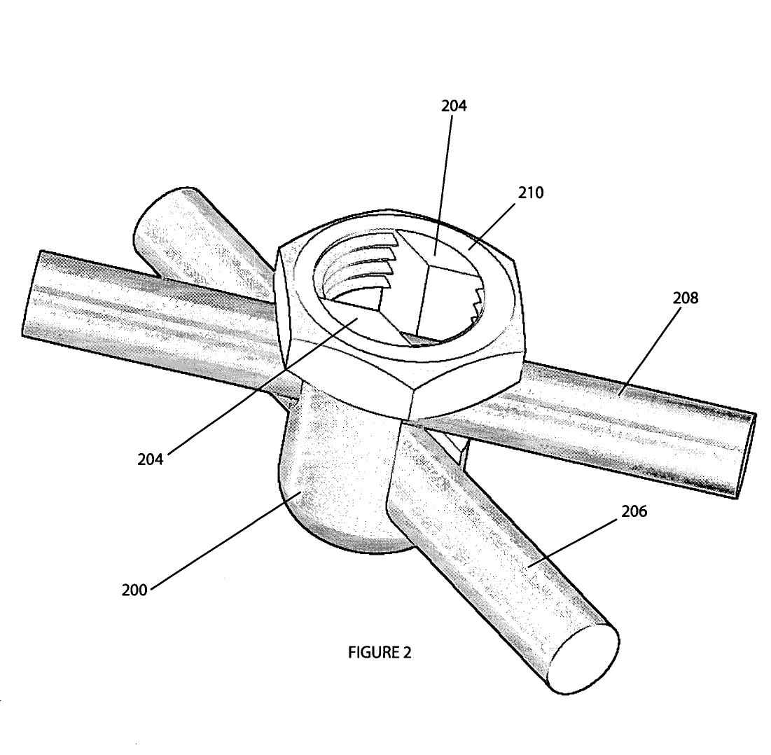

FIG. 1A shows a plurality of vertebrae 110 secured relative to one another by crossed rods 106, 108, the rods 106, 108 being maintained in their crossed disposition by a cross link 100, in accordance with an embodiment of the present invention. The rods 106, 108 are secured to the bones 110 by a manner known in the art, e.g., by using a screw and rod system of a type known in the art. Typically, for each rod extent attachment to the vertebral body, this procedure will involve pre-drilling and tapping screw holes in the pedicles of the vertebral body, threading bone screws therein respectively, mating with each bone screw head a rod receiving member that can float polyaxially relative to the bone screw head until it is locked thereto, placing a rod within the rod receiving channel of the rod receiving member, adjusting the direction of the rod as clinically desirable within the polyaxial capability of the rod receiving member, and threading a cap onto the rod receiving member to compress the rod against the bone screw head and the bone screw head against the interior of the rod receiving member to lock the rod receiving member to the bone screw head and therefore the position of the rod relative to the pedicle.

As shown in FIG. 1A, in accordance with an embodiment of the present invention, the rods 106, 108 are directed to cross one another to enhance the stability of the construct. The extents of the rods 106, 108 are secured to appropriate vertebrae 110 as clinically necessary, and a cross link 100 of the present invention is secured to the rods 106, 108 at their intersection, to structurally ensure the maintenance of the rods rods 106, 108 relative position to one another. An alternate arrangement of crossed rods 106, 107, 108 is shown in FIG. 1B, with a plurality of cross links 100 being used to secure the construct. It should be understood that in accordance with the present invention, any clinically desirable rod arrangement, with any number of cross links maintaining two or more rods in crossed disposition to one or more other rods, can be used, and the invention is not limited to the arrangements shown.

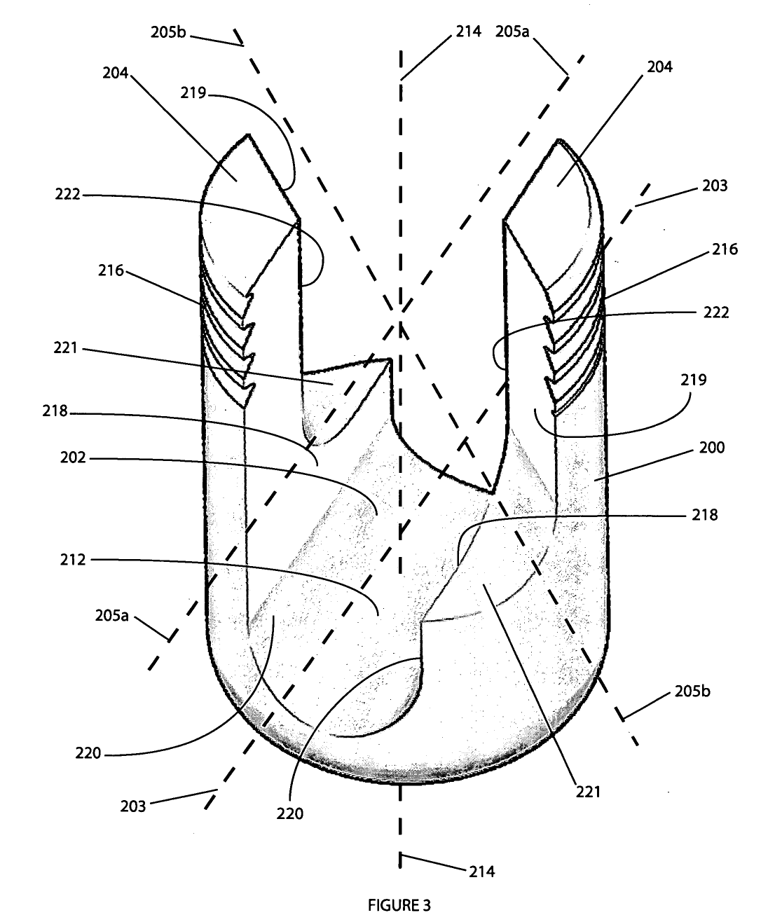

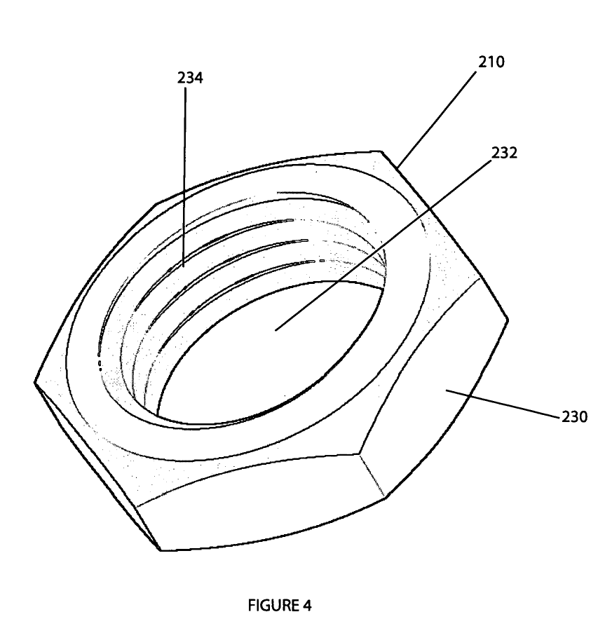

FIG. 2 is a perspective view of a first embodiment of the cross link shown in FIGS. 1A and 1B, maintaining two rods in crossed disposition. The cross link includes a rod supporting body, for example, a bolt 200. The bolt 200 has two extensions, for example, arms 204 defining a trough 202 (best shown in FIG. 3) forming a lower rod receiving channel (best shown denoted by dashes 203 on FIG. 3) and an upper rod receiving channel (best shown denoted by dashes 205a, denoting a first rod position discussed below, and 205b, denoting a second rod position discussed below, and rod positions rotatably therebetween, the upper rod receiving channel being hereinafter referred to also as 205 for simplicity). A lower rod 206 is shown in the lower channel 203 and an upper rod 208 is shown in the upper channel 205. The cross link further includes a compression providing element, for example, a nut 210 that threads around the arms 204. During assembly, the lower rod 206 is placed into the lower channel 203 and the upper rod 208 is placed in the upper channel 205 on top of the lower rod 206. (Alternatively, once the rods 206, 208 are placed by the surgeon in crossed disposition as described above, the bolt 200 can be passed under the rods 206, 208 and the arms 204 brought around the rods 206, 208 from underneath the rods 206, 208.) Thereafter, the nut 210 is threaded onto the arms 204 and tightened to compression lock the upper rod 208 against the lower rod 206, and the lower rod 206 against the floor of the trough 202. In this manner, the rods 206, 208 can be secured in crossed disposition.

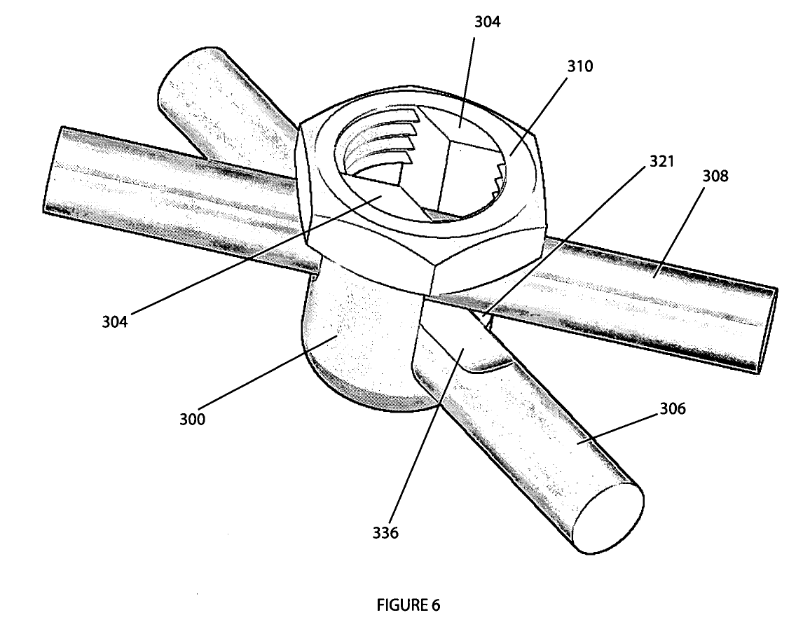

FIG. 3 is a perspective view of the bolt 200 of the cross link embodiment shown in FIG. 2. The bolt 200 has the two arms 204 extending to form the trough 202 that has a floor 212 and walls 218. Each of the arms 204 has an outwardly facing curved surface 216 that is threaded to receive the threads of the nut 210 (shown in FIG. 5). Preferably, each of the arms 204 further is positioned and dimensioned so as to allow the lower 206 and upper 208 rods to be positioned relative to one another at a variety of angles.

One example of a suitable positioning and dimensioning is illustrated and described as follows. It should be understood that alternative positionings and dimensionings are possible within the scope of the present invention, and that this illustrated example is merely one of the many possibilities. In this example, as shown, lower portions 220 of the walls 218 are parallel to form the lower channel 203 such that the lower rod 206 positioned in the lower channel 203 cannot be rotated therein about the longitudinal axis (denoted by dashes 214) of the bolt 200. Upper portions 222 of the walls 218 are angled to define the limits to which the upper rod 208 may be rotated about the longitudinal axis 214 of the bolt 200 to be positioned relative to the lower rod 206 at a variety of angles. More particularly, the upper rod 208 can be rotated to a first position 205a, to a second position 205b, or to any rotational position therebetween. (Or, in the case where the rods 206, 208 are crossed and secured at their ends to vertebral bodies, and thereafter the cross link is applied to the rods 206, 208, the walls 218 can accommodate a variety of relative angles at which the lower 206 and upper 208 rods are crossed with respect to one another.) This allows the rods 206, 208 to be positioned as clinically necessary during formation of the construct. Upon application of the nut 210 to the bolt 200, the rods 206, 208 will be locked at the desired positions. It should be noted that the shelves 221 formed by the angled upper portions 222 of the walls 218 must be at a depth that is more shallow than the diameter of the lower rod 206, so that the upper rod 208 can be compressed against the lower rod 206 without interference from the shelves 221. Accordingly, the rods 206, 208 can be locked at any angle relative to one another, limited only by the boundaries set by the walls 218. In this example, the rods 206, 208 can be rotated relative to one another up to 45 degrees, because the upper portions 222 of the walls 218 each have a surface 219 that is angled 45 degrees with respect to the corresponding lower portion 220 of the wall 218, as indicated. However, it should be understood that any desired angle can be accommodated by other embodiments of the present invention, with appropriate tailoring of the wall dimensions and/or angles, and/or the number of walls. In some embodiments, it is contemplated that the walls would be formed such that the lower 206 and upper 208 rods can be rotated about the longitudinal axis 214 of the bolt 200. For example, both rod receiving channels could be formed from angled opposing inwardly facing walls. Also, for example, the lower rod receiving channel, and not the upper rod receiving channel, could be formed from angled opposing inwardly facing walls, so that the lower rod 206 can be rotated but not the upper rod 208.

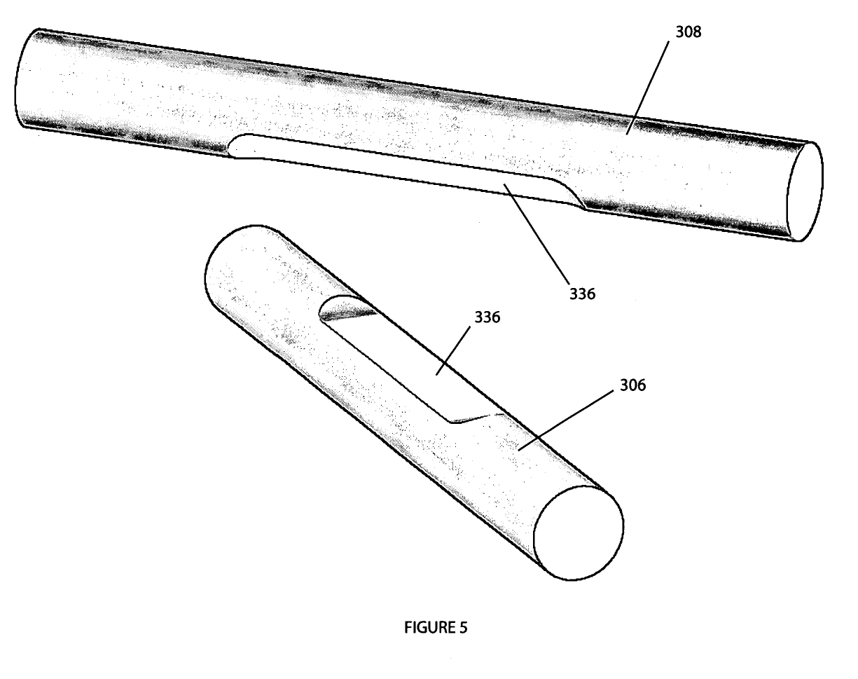

FIG. 4 is a perspective view of the nut 210 of the cross link embodiment shown in FIG. 2. The nut 210 has an angled perimeter 230 to facilitate rotation of the nut 210 onto the bolt 200 by a tool such as, for example, a wrench. The nut 210 further has a bore 232 that has a diameter encompassing the arms 204 of the bolt 200. The wall 234 of the bore 232 is threaded to be easily received by the threads on the outwardly facing curved surfaces 216 of the arms 204 of the bolt 200. As the final step of the assembly of the construct, the nut 210 is threaded around the arms 204 until the bottom of the nut 210 is compressed against the upper rod 208, the upper rod 208 is compressed against the lower rod 206, and the lower rod 206 is compressed against the floor 212 of the trough 202. The construct thereby maintains the rods 206, 208 in crossed disposition to provide additional stability to the construct.

It should be understood that the present invention encompasses embodiments having a bolt but where the first rod receiving channel is not defined by the trough, but rather is defined by a bore through the bolt body itself, which bore is not in communication with the trough. However, the second rod receiving channel would be formed by the trough so that the nut can be applied to the bolt to compression lock the second rod in the second rod receiving channel at its angulated position relative to the first rod. In such embodiments, the first rod remains free to move longitudinally in the first rod receiving channel even after the second rod has been compression locked in the second rod receiving channel. (In yet another alternative, the nut could be applied to merely prevent the second rod from escaping the second rod receiving channel, so that the second rod would also be free to move longitudinally in the second rod receiving channel as well.) Having one or both of the rods free to move longitudinally may be desirable in certain clinical applications.

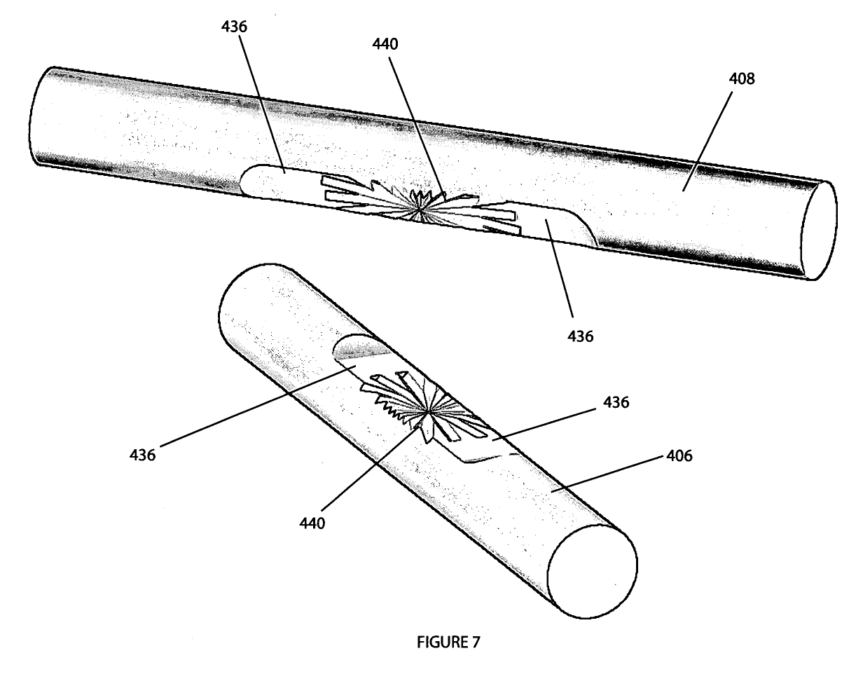

With regard to a second embodiment of the cross link shown in FIG. 1, FIG. 5 is a perspective view of alternate lower 306 and upper 308 rods of the present invention, each having a securing feature on the surface against which the other rod is to be compressed. While any suitable securing feature can be used with, and is contemplated by the present invention, the securing feature in this example is a flattened surface 336. The flattened surface 336 is a securing feature in that it provides a greater amount of surface area against which the other rod can be compressed when locked in the cross link of the present invention, compared to the merely tangential surface area provided by the fully cylindrical rods that are shown in FIG. 2. It should be understood that a cross link bolt suitable for use with these alternate rods 306, 308 would be dimensioned differently than the bolt 200 of the first cross link embodiment shown in FIG. 2. In particular, the height of the shelves above the floor of the trough must be smaller than the thickness of the lower rod 306 (at the section of the lower rod 306 having the securing feature) from the lateral center of the flattened surface 336 to the tangent point 338 on the curved surface of the lower rod 306 opposite the flattened surface 336. Accordingly, crossed alternate lower 306 and upper 308 rods are shown in FIG. 6, being secured by an alternate rod supporting body, for example, an alternate bolt 300 formed similarly to the bolt 200 but with this change to the height of the shelves 321. Preferably, as shown, the flattened surfaces 336 are roughened so as to provide even greater fixation when compressed against one another. Features of this second embodiment of the cross link that are similar to those of the first embodiment are similarly referenced, but in the 300s rather than the 200s.

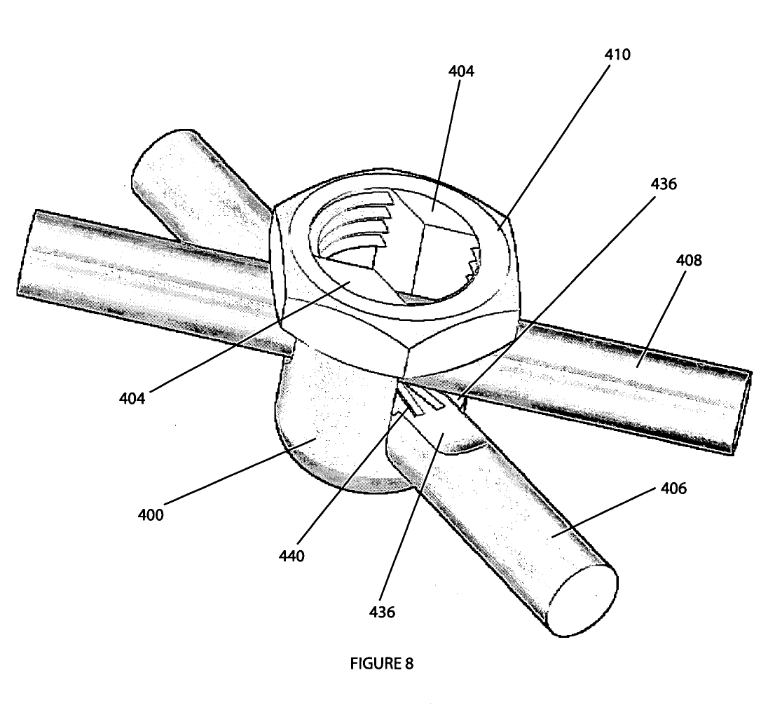

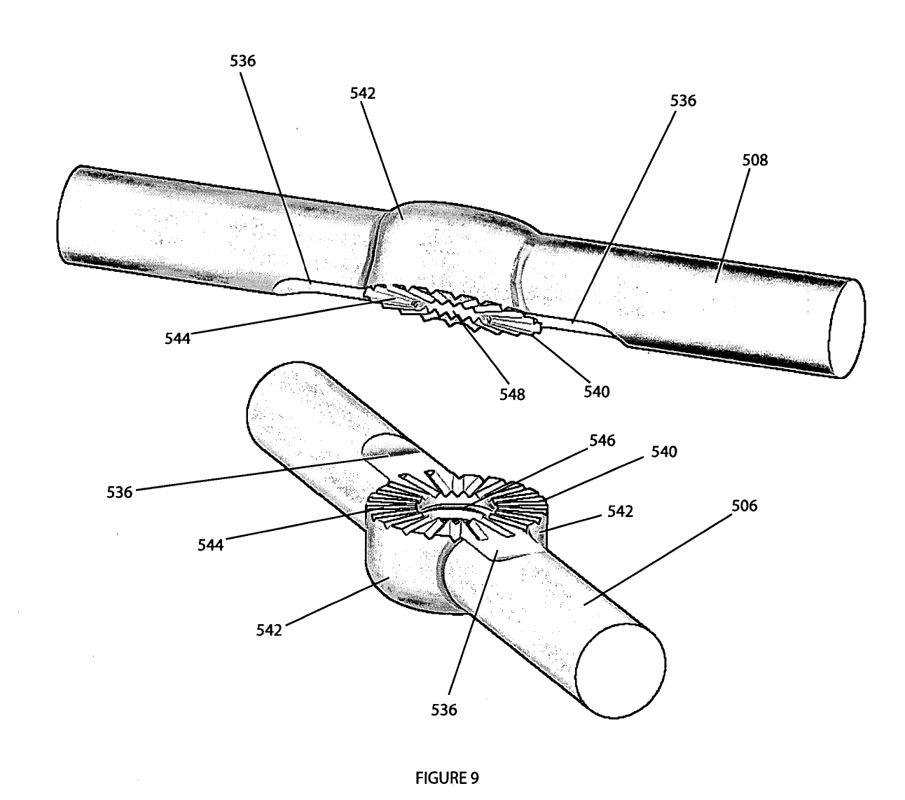

With regard to a third embodiment of the cross link shown in FIG. 1, FIG. 7 is a perspective view of other alternate lower 406 and upper 408 rods of the present invention, each having a securing feature on the surface against which the other rod is to be compressed. Again, while any securing feature can be used with, and is contemplated by, the present invention, the securing feature in this example is a flattened surface 436 having at least one protrusion, for example, a plurality of radially extending ridges 440. The flattened surface 436 having radially extending ridges 440 is a securing feature in that it provides a greater amount of surface area against which the other rod can be compressed when locked in the cross link, compared to the merely tangential surface area provided by the fully cylindrical rods that are shown in FIG. 2. Further, the radially extending ridges 440 will interlock when the rods 406, 408 are compressed against one another, providing even greater fixation. As with the alternate bolt 300 on FIG. 6, it should be understood that a cross link bolt suitable for use with these other alternate rods 406, 408 would be dimensioned differently than the bolt 200 of the first cross link embodiment shown in FIG. 2. In particular, the height of the shelves above the floor of the trough must be smaller than the thickness of the lower rod 406 (at the section of the lower rod 406 having the securing feature) from the lateral center of the floor of the flattened surface 436 to the tangent point 438 on the curved surface of the lower rod 406 opposite the flattened surface 436. Accordingly, crossed alternate lower 406 and upper 408 rods are shown in FIG. 8, being secured by another alternate rod supporting body, for example, another alternate bolt 400 formed similarly to the bolt 200 but with this change to the height of the shelves 421. Features of this third embodiment of the cross link that are similar to those of the first embodiment are similarly referenced, but in the 400s rather than the 200s.

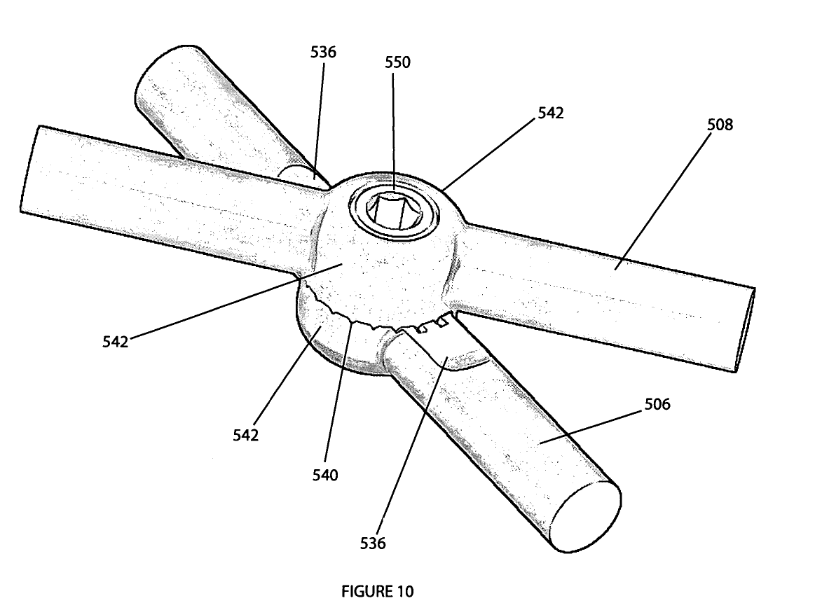

With regard to a fourth embodiment of the cross link shown in FIG. 1, FIG. 9 is a perspective view of still other alternate lower 506 and upper 508 rods of the present invention, each having a securing feature on the surface against which the other rod is to be compressed. Again, while any securing feature can be used with, and is contemplated by, the present invention, the securing feature in this example is a flattened surface 536 having at least one protrusion, for example, a plurality of radially extending ridges 540. The flattened surface 536 having radially extending ridges 540 is a securing feature in that it provides a greater amount of surface area against which the other rod can be compressed, compared to the merely tangential surface area provided by the fully cylindrical rods that are shown in FIG. 2. Further, the radially extending ridges 540 will interlock when the rods 506, 508 are compressed against one another, providing even greater fixation. To further increase the surface area against which the other rod can be compressed, the securing feature is enhanced in that the flattened surface 536 laterally extends, as shown, beyond the boundaries of the cylinder defined by the rod body, the extensions 542 curvately joining the rod body to form a central flattened circular region 544 where the laterally extending ridges 540 are disposed. Accordingly, when the rods 506, 508 are mated at their central circular regions and compressed against one another, the large surface areas on which the ridges 540 interlock provides greater fixation than that achieved by compressing together traditional rods 206, 208 shown in FIG. 2.

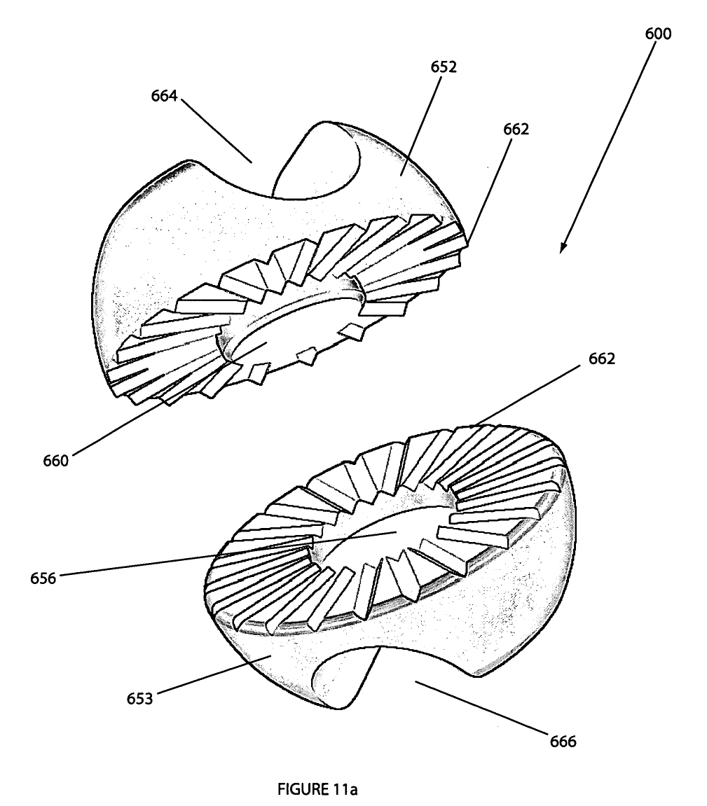

With additional reference to FIG. 10, which is a perspective view of the rods 506, 508 compressed together, the rods 506, 508 in this fourth embodiment are compressible against one another because the central circular regions 544 are each provided with a central bore 546, 548 that passes through the rod 506, 508, and a compression providing element, for example, a set screw 550 is provided to pass through the bores 546, 548. The central bore 546 of the lower rod 506 is threaded, and the central bore 548 of the upper rod 508 is smooth but accommodates the greater diameter of the threaded portion of the screw 550, so that when the screw 550 is passed through the bores 546, 548 and rotated within the threads of the central bore 546 of the lower rod 506, the lower rod 506 is brought to compress against the upper rod 508. Accordingly, central circular regions 544 come together, and the ridges 540 interlock, so that as the regions are compressed together, a compression lock is secured with a fixation greater than that achieved by compressing together traditional rods 206, 208 shown in FIG. 2.

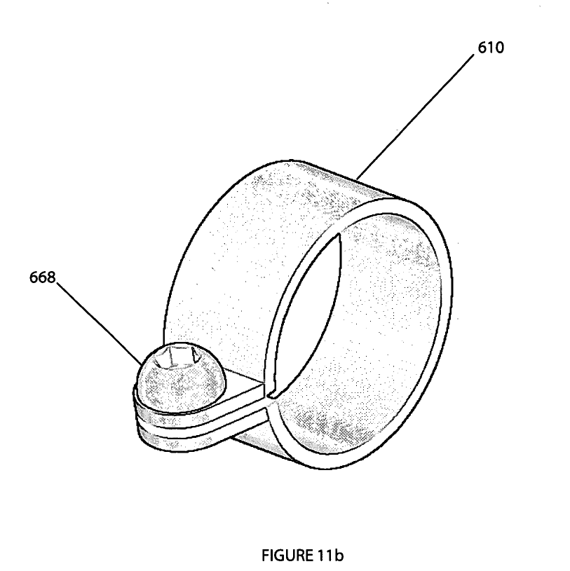

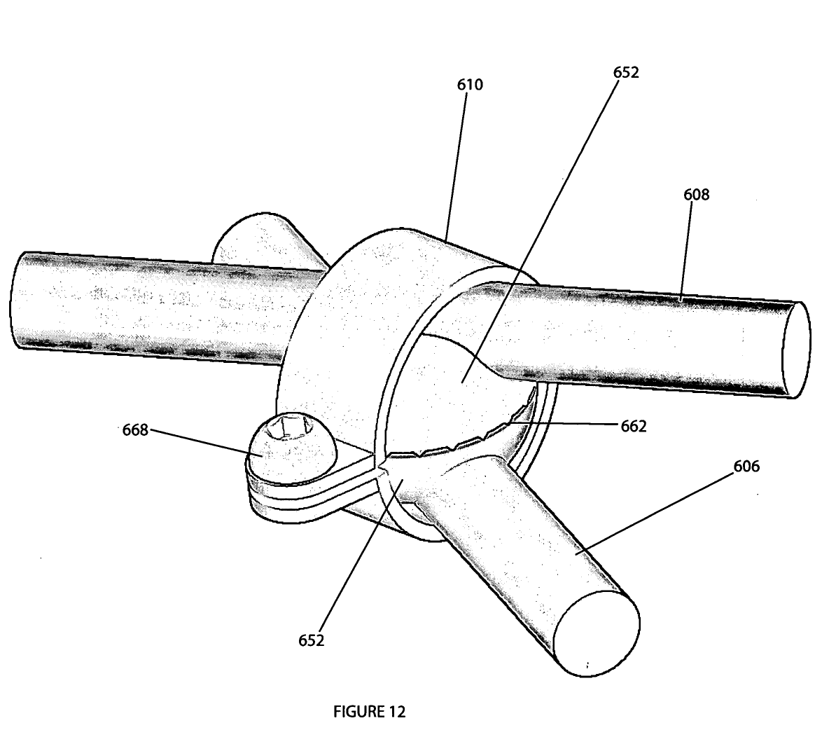

With regard to a fifth embodiment of the cross link shown in FIG. 1, FIG. 11A is a perspective view of a rod supporting body, for example, a dual rod holder 600, which in cooperation with a compression providing element, for example, a clamp 610 shown in perspective in FIG. 11B is adapted to hold in crossed disposition traditional rods 606, 608 which are similar to the rods 206, 208 shown in FIG. 2. FIG. 12 is a perspective view of exemplary rods 606, 608 being held in crossed disposition by this fifth embodiment of the cross link shown in FIG. 1. More particularly, the dual rod holder 600 includes two body portions, for example, two hemispherical members 652, 653 rotationally mounted to one another at their flat sides, so that they collectively form a sphere that has hemispherical sections 652, 653 that can swivel relative to one another about a polar axis of the sphere. While any rotational coupling can be used with, and is contemplated by, the present invention, the rotational coupling in this example includes a central bore 656 in one of the hemispheres 652 at its flat side, and a central post 660 extending from the flat side of the other hemisphere 653, with the post 660 fitting for free rotation within the central bore 656.

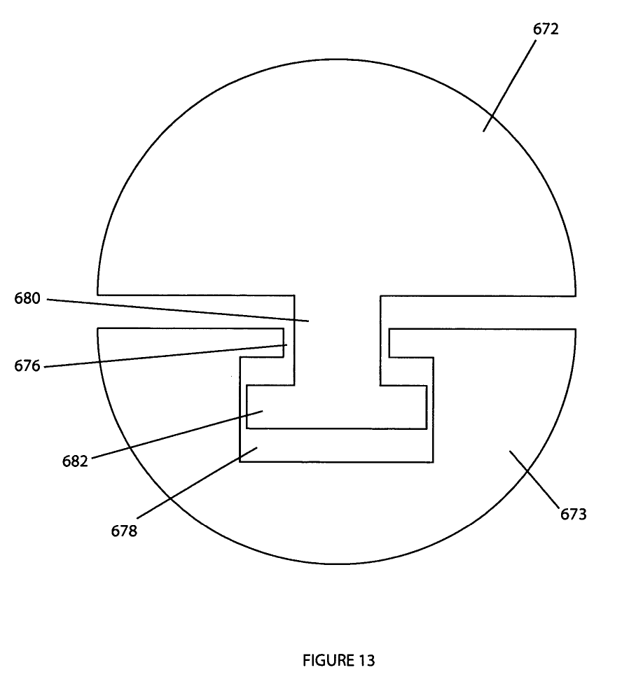

It should be understood that the present invention encompasses embodiments where the hemispheres are prevented from separating in tension by the rotational mounting. While such functionality can be accomplished in many ways, one example of such a tension bearing rotational mounting is illustrated in a cut away view of, alternate hemispheres 672, 673 in FIG. 13, which are similar to the hemispheres 652, 653 of FIG. 12, but have a different bore and a different post. More particularly, the bore 676 of the alternate hemisphere 672 leads to a cavern 678 in the hemisphere 672, the cavern 678 having a larger diameter than the bore 676, and the post 680 of the other hemisphere 673 has a flanged end that forms a generally cylindrical head 682 that fits for free rotation in the cavern 678. The head 682 is prevented from exiting the cavern 678 because while the post 680 has a diameter fitting in the bore 676, the 682 head has a larger diameter than the bore 676. Accordingly, the hemispheres 672, 673 can rotate freely with respect to one another, and cannot fully separate because the cylindrical head 682 cannot escape the cavern 678 through the bore 676.

Referring back to FIG. 12, preferably each of the hemispheres 652, 653 has a rod receiving channel 664, 666 in which the rods 606, 608 can be placed. Once the rods 606, 608 are placed in the channels 664, 666, they can be angled with respect to one another by a relative rotation of the hemispheres 652, 653. Once the desired angle has been reached for the particular clinical application, the clamp 610 applied around the hemispheres 652, 653 to encompass the rods 606, 608 as shown in FIG. 12 fixes the hemispheres 652, 653 at their positions relative to one another, and fixes the rods 606, 608 in the channels 664, 666 so that they are secured in cross disposition. While the clamp 610 can be secured to itself by any method or device, the example shown uses a clamp having bored ends, and a securing screw 668 passing through the bores to hold the ends together as shown. The mating surfaces of the hemispheres 652, 653 can be provided with a roughened surface and/or at least one protrusion (for example, a plurality of ridges 668 such as those as described above on the rods in the other embodiments) to enhance the fixation of the hemispheres 652, 653 to one another.

It should be understood that the present invention encompasses embodiments having the rotatable mounted body portions, but where one or both of the rod receiving channels are not defined by a trough in the body portion, but rather is defined by a bore through the body portion. In such embodiments, a rod in such a bore would remain free to move longitudinally therein even after the body portions have been compression locked together and thereby prevented from rotating with respect to one another. Having one or both of the rods free to move longitudinally may be desirable in certain clinical applications.

It should also be understood that for any of the embodiments, not only may the rods have roughened or featured areas at their contact surfaces to enhance their compression locking to one another, but also the surfaces of one or both of the rod receiving channels may be roughened or featured to enhance the compression locking of the rods in those channels.

In accordance with a method of the present invention, an immobilization assembly can be stabilized on the spine, preferably using the devices described herein. As one example of an appropriate clinical application in which the system and method of the present invention is useful, a patient presenting an L-4/L-5 degenerative spondylolisthesis receives the necessary laminectomy, but the stabilization rods, rather than being positioned in parallel on either side of the spinous process and being connected by a transverse connector, are set in crossed disposition. When necessary, this can be facilitated by the use of polyaxial screws with one pair of screws left relatively proud to allow the rod they support to cross over the other rod. A cross link of the present invention is applied to the rods before or after the setting of the rods in crossed disposition. Depending on the type of cross link used, it will be easier for the surgeon to apply the cross link to the rods before setting them, or to apply the cross link to the rods after setting them. In the case where the cross link is formed by modified rods (e.g., as in the fourth embodiment described above), the rods would typically be set in crossed disposition, then compressed together (e.g., by applying the screw to the bored central portions of the rods) to stabilize the construct. The soft tissues of the back can be sutured down onto the rods, which minimizes dead space and therefore reduces post-operation bleeding. Advantages of the method of the present invention include that the resulting fusion is easier to grade radiographically because the rods do not hide the area of interest, and that if reoperation is required, scar revision is easier because a depth and plane of resection are already defined. While the procedure has been described for use with the laminectomized spine, the system and method of the present invention are useful in other clinical applications as well. For example, many lumbar spinal fusions are performed for degenerative conditions, which often require a laminectomy. Thus, the present invention can be applied universally to all implant systems. As described below, single and multiple level constructs requiring in situ fixation are among the preferred applications.

While there have been described and illustrated specific embodiments of the invention, it will be apparent to those skilled in the art that variations and modifications are possible without deviating from the broad spirit and principle of the invention. The invention, therefore, shall not be limited to the specific embodiments discussed herein.

What is claimed is:

1. A cross link for maintaining at least two spine stabilization rods in crossed disposition, the cross link comprising:
a rod supporting body having a first rod receiving channel in which a first spine stabilization rod is dispositionable, and a second rod receiving channel in which a second spine stabilization rod is dispositionable and when therein is angulatable, through a plurality of angles, relative to the first spine stabilization rod when the first spine stabilization rod is disposed in the first rod receiving channel; and
at least one compression providing element that is applicable to the rod supporting body to compression lock the first spine stabilization rod in the first rod receiving channel and to compression lock the second spine stabilization rod in the second rod receiving channel and to compressively fix the angle at which the second spine stabilization rod is angulated with respect to the first spine stabilization rod.

2. The cross link of claim 1, wherein the rod supporting body includes two extensions that by their locations relative to one another together define a trough, the trough forming the first rod receiving channel through a first portion of the trough, the trough forming the second rod receiving channel through a second portion of the trough.

3. The cross link of claim 2, wherein the first portion of the trough is defined by at least one set of opposing parallel surfaces forming the first rod receiving channel, and the second portion of the trough is defined by at least two sets of opposing parallel surfaces forming the second rod receiving channel and providing at least two possible rod positions within the second rod receiving channel, at least one of the possible rod positions being occupiable by the second rod so that the second rod is angulated relative to the first rod when the first rod is in the first rod receiving channel.

4. The cross link of claim 2, wherein the compression providing element is applicable to the extensions, when the rods are in the rod receiving channels, to bear against one of the rods to cause the one of the rods to bear against the other rod to compression lock the rods against one another and in the trough.

5. The cross link of claim 4, wherein at least one of the extensions has threads and the compression providing element is threaded to cooperate with the threads of the at least one extension, such that the compression element is, when the rods are in the rod receiving channels, threadable with the at least one extension to bear against the one of the rods.

6. The cross link of claim 1, wherein the rod supporting body comprises first and second body portions, the first and second body portions being rotatably mounted to one another, the first body portion having the first rod receiving channel, the second body portion having the second rod receiving channel.

7. The cross link of claim 6, wherein the first and second body portions are each hemispherical to provide a flat surface thereof and a curved surface thereof, the first and second body portions being rotatably mounted to one another at their flat surfaces, the curved surface of the first body portion having the first rod receiving channel, the curved surface of the second body portion having the second rod receiving channel.

8. The cross link of claim 7, wherein the compression providing element is securable around the first and second body portions to encompass the first and second body portions and the first and second rods in the first and second rod receiving channels, to compression lock the first rod in the first rod receiving channel and to compression lock the second rod in the second rod receiving channel and to compression lock the first and second body portions to one another so that they are not rotatable with respect to one another.

9. The cross link of claim 8, wherein the compression providing element includes a circular clamp having first and second ends that are securable to one another.

10. A spine stabilization assembly for maintaining at least two spine stabilization rods in crossed disposition, the assembly comprising:
a first spine stabilization rod, the first spine stabilization rod extending longitudinally and having two ends, each of the ends being suitable for fixation to a vertebral body of the spinal column; and
a second spine stabilization rod, the second spine stabilization rod extending longitudinally and having two ends, each of the ends being suitable for fixation to a vertebral body of the spinal column,
the first and second spine stabilization rods being suitably sized for implantation adjacent a spinal column in crossed disposition,
each of the first and second spine stabilization rods having a bore through a central portion of the rod, the rods being compressible against one another at their central portions in crossed disposition relative to one another; and
a compression providing element that is applicable to the rods through the bores to compress the rods together in crossed disposition;
whereby the assembly is useful for stabilizing a portion of the spinal column.

11. The spine stabilization assembly of claim 10, wherein the bore of the first rod has a smooth inner surface and the bore of the second rod has a threaded inner surface, and the compression providing element includes a threaded screw fittable in the bores and engageable with the threads of the bore of the second rod, such that a passing of the screw through the bore of the first rod and into the bore of the second rod and a rotation of the screw thereafter urges the second rod against the first rod to compress the rods together.

12. The spine stabilization assembly of claim 10, wherein the central portion of each rod has a cross-section having a straight edge, thereby forming a flattened central region of the rod, the flattened central region providing an increased surface area against which the other rod is compressible.

13. The spine stabilization assembly of claim 10, wherein the flattened central region of each rod includes a plurality of protrusions thereon, the central region thereby providing an area with which the central region of the other rod is interlockable when compressed thereagainst.

14. The spine stabilization assembly of claim 13, wherein the plurality of protrusions includes a plurality of ridges extending radially toward an outer edge of the rod.

15. The spine stabilization assembly of claim 12, wherein the central region of each rod is laterally enlarged.

16. The spine stabilization assembly of claim 15, wherein the flattened central region of each rod includes a plurality of protrusions thereon, the central region thereby providing an area with which the central region of the other rod is interlockable when compressed thereagainst.

17. The spine stabilization assembly of claim 16, wherein the plurality of protrusions includes a plurality of ridges extending radially toward an outer edge of the rod.

18. A spine stabilization assembly for maintaining at least two spine stabilization rods in crossed disposition, the assembly comprising:
a first spine stabilization rod, the first spine stabilization rod extending longitudinally and having two ends, each of the ends being suitable for fixation to a vertebral body of the spinal column; and
a second spine stabilization rod, the second spine stabilization rod extending longitudinally and having two ends, each of the ends being suitable for fixation to a vertebral body of the spinal column,
the first and second spine stabilization rods being suitably sized for implantation adjacent a spinal column in crossed disposition;
a rod supporting body having a first rod receiving channel in which the first spine stabilization rod is dispositionable, and a second rod receiving channel in which the second spine stabilization rod is dispositionable and when therein is angulatable, through a plurality of angles, relative to the first spine stabilization rod when the first spine stabilization rod is disposed in the first rod receiving channel; and
a compression providing element that is applicable to the rod supporting body to compression lock the first spine stabilization rod in the first rod receiving channel and to compression lock the second spine stabilization rod in the second rod receiving channel and to compressively fix the angle at which the second spine stabilization rod is angulated with respect to the first spine stabilization rod.

19. The cross link of claim 18, wherein the rod supporting body includes two extensions that by their locations relative to one another together define a trough, the trough forming the first rod receiving channel through a first portion of the trough, the trough forming the second rod receiving channel through a second portion of the trough.

20. The cross link of claim 18, wherein the rod supporting body comprises first and second body portions, the first and second body portions being rotatably mounted to one another, the first body portion having the first rod receiving channel, the second body portion having the second rod receiving channel.
